(19) United States
(12) Patent Application Publication
(10) Pub. No.: US 2018/0333398 A1
(43) Pub. Date: Nov. 22, 2018

(54) METHOD OF WEIGHT REDUCTION

(71) Applicant: NAIA METABOLIC, INC., Richmond, CA (US)

(72) Inventors: Ken Walder, Ocean Grove (AU); Guy Krippner, Newtown (AU); Geoff Nicholson, Geelong (AU)

(21) Appl. No.: 15/892,989

(22) Filed: Feb. 9, 2018

Related U.S. Application Data
(63) Continuation of application No. 14/403,495, filed on Nov. 24, 2014, now abandoned.
(60) Provisional application No. 61/651,335, filed on May 24, 2012.

(51) Int. Cl. A61K 31/433; A61K 31/155

(52) U.S. Cl. CPC A61K 31/433; A61K 31/155; A61K 2300/00

(57) ABSTRACT

The present disclosure relates generally to the use of methazolamide in therapy. The disclosure particularly relates to regulation of glucose homeostasis and reduction of body mass in patients suffering from or susceptible to diseases and associated conditions, in which undesirably high blood glucose levels are involved or implicated, such as diabetes, syndrome X, hyperglycaemia, vascular disease and kidney disease. The present disclosure further relates to compounds and agents and compositions thereof for use in the treatment methods.

### FIELD

The present disclosure relates generally to the use of methazolamide in therapy. The disclosure particularly relates to reduction of body mass in patients suffering from or susceptible to diseases and associated conditions, in which undesirably high blood glucose levels are involved or implicated. The present disclosure further relates to compounds and agents and compositions thereof for use in the therapy.

### DESCRIPTION OF THE PRIOR ART

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Glucose is the body's preferred energy source. Blood glucose is derived from a combination of glucose absorbed from the diet and glucose produced by the liver and released into the blood stream (hepatic glucose production). Once entered into the blood stream, glucose requires the assistance of insulin to enter hepatic, muscle and adipose cells in order to be stored or utilised. Another major action of insulin is to suppress hepatic glucose production. In a healthy individual, glucose homeostasis is controlled primarily by insulin. As blood glucose levels rise, such as after eating, specialised β-cells within the pancreas release insulin which suppresses hepatic glucose production and promotes glucose uptake, intracellular metabolism and glycogen synthesis by the body's target tissues. Thus, in healthy individuals, blood glucose concentrations are strictly controlled, typically in the range of 80-110 mg/dl. However, where the pancreas produces an inadequate insulin response, or the target cells do not respond appropriately to the insulin produced, this results in a rapid accumulation of glucose in the blood stream (hyperglycemia).

High blood glucose levels over time may cause cardiovascular disease, retinal damage, renal failure, nerve damage, erectile dysfunction and gangrene (with the risk of amputation). Furthermore, in the absence of available glucose, cells turn to fats as an alternative energy source. Resulting ketones, a product of fat hydrolysis, can accumulate in the blood stream instigating hypotension and shock, coma and even death.

Diabetes is a metabolic disorder characterized by chronically elevated blood glucose levels (greater than about 126 mg/dL or 7.0 mmol/L) from either inadequate insulin secretion (Type 1 diabetes) and/or an inadequate response or sensitivity of body tissues to insulin action (Type 2 diabetes). One of the primary diagnostic features of diabetes is the individual's loss of control over glucose homeostasis, so that post-prandial blood glucose levels remain elevated after meals and may remain high for extended periods of time. Diabetes may be characterised by persistent hyperglycemia, polyuria, polydipsia and/or hyperphagia, chronic microvascular complications such as retinopathy, nephropathy and neuropathy, and macrovascular complications, such as hyperlipidemia and hypertension which can lead to blindness, end-stage renal disease, limb amputation and myocardial infarction. High blood glucose levels and insulin resistance are also associated with fatty liver disease, which can progress to chronic inflammation, fibrosis and cirrhosis.

The three most common types of diabetes are type 1, type 2 and gestational.

Type 1 diabetes, known as insulin dependent diabetes mellitus (IDDM), or juvenile-onset diabetes, accounts for 10-15% of all diabetes cases. It is most commonly diagnosed in children and adolescents but can occur in young adults as well. It is characterised by β-cell destruction resulting in a loss of insulin secretory function. Most cases relate to autoimmune destruction of the β-cells. Treatment is via insulin injection and must be continued indefinitely.

Type 2 diabetes, known as non-insulin dependent diabetes mellitus (NIDDM) or late-onset diabetes, insulin levels are initially normal but the body's target cells lose their responsiveness to insulin. This is known as insulin resistance or insulin insensitivity. To compensate for this resistance, the pancreas secretes excess insulin. Over time, the pancreas becomes less able to produce enough insulin, resulting in chronic hyperglycemia. Initial symptoms of type 2 diabetes are typically milder than for type 1 and the condition may go undiagnosed for many years before more severe symptoms are observed. Lifestyle (smoking, poor diet and inactivity) is considered to be the major determinant of type 2 diabetes incidence, although a genetic predisposition increases the risk of developing this disease.

Gestational diabetes occurs in about 2-5% of all pregnancies. It is temporary, but if untreated may cause foetal complications. Most sufferers make a complete recovery after the birth. However, a proportion of women who develop gestational diabetes go on to develop type 2 diabetes.

Other, less common, causes of diabetes include genetic defects in β-cells, genetically related insulin resistance, diseases of the pancreas, hormonal defects, malnutrition and chemical or drug influences.

Impaired glucose tolerance and impaired fasting glucose, are pre-type 2 diabetic states, closely related to type 2, and occur when the blood glucose level is higher than normal, but not high enough to be classified as diabetes (about 100-125 mg/dL; 5.6-6.9 mmol/L). As with type 2 diabetes, the body produces insulin but in an insufficient amount or the target tissues are unresponsive to the insulin produced.

Impaired glucose tolerance, impaired fasting glucose and insulin resistance are components of Syndrome X, also known as Insulin Resistance Syndrome (IRS) or metabolic syndrome, which is a cluster of risk factors for heart disease that also includes: obesity, atherosclerosis, hypertriglyceridemia, low HDL cholesterol, hyperinsulinemia, hyperglycemia and hypertension. It is therefore apparent that insulin resistance, or insensitivity, can play a significant role in diabetes and other hyperglycemia-related conditions.

The prevalence of type 2 diabetes has more than doubled over the last 2 decades and continues to grow at an alarming rate. The World Health Organization (WHO) estimates that 346 million people worldwide suffer from type 2 diabetes (approximately 4.9% of the world's population) with at least 50% of the diabetic population unaware of their condition (World Health Organization. Diabetes. Fact sheet No 312 August 2011, (www.who.int)). Another 7 million people are estimated to become diabetic each year. The increase in diabetes incidence worldwide is a particular concern in children: type 2 diabetes was diagnosed in 1-2% of children 30 years ago, but accounts for up to 80% of pediatric diabetes cases reported today. India currently has the highest number of diabetic persons, followed by China, the USA, Russia and Germany. Approximately 1.7 million Australians (7.5% of the population) have type 2 diabetes and 275 Australian adults become diabetic every day. Another 2 million Australians have pre-diabetes and are at risk of developing type 2 diabetes (Diabetes Australia—Vic (www.diabetesvic.org.au/health-professionals/diabetes-facts)). In the United States, an estimated 25.8 million people (8.3% of the population) have diabetes and a further 79 million are prediabetic (U.S. Department of Health and Human Services, Centers for Disease Control and Prevention (2011). National diabetes fact sheet: national estimates and general information on diabetes and prediabetes in the United States (www.cdc.gov/diabetes)). 1.9 Million new cases of adult diabetes are diagnosed in the US each year and at least one prediction has indicated that the current growth in diagnosed and undiagnosed diabetes means 50% of the US population could be diabetic or prediabetic by 2020 (UnitedHealth Group's Center for Health Reform & Modernization. The United States of Diabetes. Working paper 5. November, 2010). The economic costs of diabetes and related conditions are dramatic. The estimated direct and indirect costs of diabetes to the Australian healthcare system are estimated to be at least AUD 3 billion. This is dwarfed by the US, where direct costs of diabetes were estimated to be USD 116 billion in 2007, with indirect costs accounting for an additional USD 58 billion. If the predicted increase in diabetes incidence in the US continues, the healthcare costs could reach USD 3.35 trillion (at least 10% of total health care spending).

Type 2 diabetes is ideally treated by lifestyle modification, particularly diet and exercise. Comprehensive clinical and epidemiological studies have demonstrated that weight loss of 5-11 kg can reduce diabetes risk by 50% and weight loss of ≥10 kg is associated with 30-40% decrease in diabetes-related deaths. Weight loss of 20-30 kg is curative of diabetes and hypertension in many patients (Labib M., (2003) The investigation and management of obesity. J Clin Pathol. 56: 17-25).

Unfortunately, most patients cannot sustain such lifestyle modifications and pharmacological intervention is required for adequate glucose control. International treatment guidelines now include metformin with diet and exercise as the first-line therapy for type 2 diabetes (Inzucchi S E et al. (2012) Medical management of hyperglycemia in type 2 diabetes: a patient-centered approach. Position statement of the American Diabetes Association (ADA) and the European Association for the Study of Diabetes (EASD). Diabetes Care 35: 1364-79; e-published ahead of print, 19 Apr. 2012). The multi-factorial nature of diabetes pathology means most patients will progress to combination therapy to maintain effective glucose control over their lifetime. If metformin and lifestyle modification are insufficient to establish glucose control, addition of a sulfonylurea, DPP4 inhibitor (such as sitagliptin), GLP-1 agonist (such as liraglutide) (second line) or three drug combinations (third line) are indicated. The thiazolidinedione (TZD) insulin sensitizers rosiglitazone and pioglitazone had previously been recommended as second-line therapy; however, significant safety concerns have severely limited their current use. Patients who cannot maintain glucose control with combination therapies will ultimately be required to use insulin. While insulin has previously been considered a last-line of diabetes therapy, physicians have become more willing to add basal insulin as a second-line therapy.

Current diabetes treatments are often limited by poor safety profiles. First-line therapy metformin causes gastrointestinal side-effects including dose-limiting diarrhea. Second-line therapy sulfonylureas (which increase insulin secretion), along with meglitinides, can cause dangerous hypoglycemia and accelerate pancreatic β-cell destruction. The sulfonylureas, meglitinides and metformin are all subject to tolerance and loss of efficacy over time. The TZD insulin sensitizers have been associated with severe edema, weight gain, bone fractures, cardiovascular side-effects (including increased risk of mortality from myocardial infarction), bladder cancer and increased risk of diabetic macular edema. Safety warnings have been issued for the DPP4 inhibitor sitagliptin regarding acute pancreatitis and the potentially fatal allergic reaction Stevens-Johnson Syndrome. The related molecule vildagliptin has been shown to elevate liver enzyme levels. Treatment with the GLP-1 agonist exenatide can cause nausea, pancreatitis and hypoglycemia. Development of antibodies to exenatide can also limit its utility in some patients. The GLP-1 agonist liraglutide has a high incidence of gastrointestinal side effects (including nausea and vomiting) and causes dose-dependent and treatment-duration-dependent thyroid C-cell tumors at clinically relevant exposures in rats and mice. Cost is also a significant issue with newer therapies. For example, sitagliptin is no more effective than metformin at lowering blood glucose levels but is 20-times more expensive (VanDeKoppel S et al. (2008) Managed care perspective on three new agents for type 2 diabetes. J Manag Care Pharm 14: 363-80.)

The limitations identified for current non-insulin diabetes medicines means there is a pressing need to develop cost-effective new therapies with improved safety and efficacy profiles; high patient compliance; and potential to maintain/improve β-cell function and delay secondary treatment failure. There is a particularly a need for new, safe insulin sensitizers to replace the TZDs. In addition, there is a therapeutic and regulatory mandate for new diabetes therapies to deliver improvements in key health parameters, ideally body weight reductions and improvements in cardiovascular health (US FDA. Guidance for Industry: Diabetes Mellitus—Evaluating Cardiovascular Risk in New Antidiabetic Therapies to Treat Type 2 Diabetes. December, 2008). Recently, WO2008/089521 disclosed the use of methazolamide (a medicine originally approved to treat glaucoma) for the treatment of diabetes and other pre-diabetic conditions. Preclinical studies have established that methazolamide is a new class of insulin sensitizer. The present disclosure describes a novel and unexpected effect of methazolamide to reduce body weight in diabetes patients.

### SUMMARY

It has now been surprisingly observed that where a patient has previously commenced treatment with an anti-diabetic agent, such as metformin, subsequent co-administration of methazolamide results in additional and unexpected weight loss. Surprisingly, this effect was not observed in patients who had not previously been using anti-diabetic agents. The present disclosure relates to the use of methazolamide in effecting weight loss in patients established on anti-diabetic or blood glucose-regulating treatment. In some embodiments the use of methazolamide may advantageously therefore be a useful adjunctive treatment for patients already established on an anti-diabetic agent treatment, by ameliorating insulin resistance, augmenting blood glucose control and reducing body mass. A reduction in body mass may allow for a reduction in the required doses of anti-diabetic or other therapeutic medicines.

Thus, the present disclosure relates to a method of effecting weight loss in a patient previously commenced on and undergoing treatment with an anti-diabetic agent, said method comprising the step of further administering methazolamide to said patient.

In a further embodiment, the disclosure relates to a method of effecting weight loss in a patient comprising:

(i) commencing treatment with an anti-diabetic agent;

(ii) continuing treatment with the anti-diabetic agent; and

(iii) subsequently commencing additional treatment with, methazolamide.

In some embodiments, the treatment with methazolamide is commenced once the patient's blood glucose levels are stabilised by the anti-diabetic agent.

The disclosure also relates to methazolamide for use in effecting weight loss in a patient previously commenced on and undergoing treatment with an anti-diabetic agent.

The present disclosure further relates to compositions for effecting weight loss in a patient previously commenced on and undergoing treatment with an anti-diabetic agent, said composition comprising methazolamide, together with one or more pharmaceutically acceptable additives.

The present disclosure also relates to the use of methazolamide in the manufacture of a medicament for effecting weight loss in a patient previously commenced on and undergoing treatment with an anti-diabetic agent.

The present disclosure also relates to a combination for use in effecting weight loss in a patient previously commenced on and undergoing treatment with an anti-diabetic agent, said combination comprising methazolamide and an anti-diabetic agent.

In some embodiments, the methazolamide is administered in an amount less than 100 mg per day, such as 90, 80, 70 60 or 50 mg per day.

In some embodiments, the anti-diabetic agent is an insulin sensitiser, such as metformin, or a pharmaceutically acceptable salt thereof.

In some embodiments, the methazolamide and anti-diabetic agents are administered orally, either simultaneously or separately.

In some embodiments the patient has a BMI of at least 25.

In some embodiments, the patient has a waist measurement of greater than 94 cm (adult men) or greater than 80 cm (adult women).

### DESCRIPTION OF THE FIGURES

FIG. 1 graphically depicts the effect of contemporaneous methazolamide treatment in reducing the body weight of diabetes patients who have been stable on metformin for at least 3 months prior to methazolamide treatment. Patients treated with methazolamide and metformin lost 2% of their starting body weight over the 24 week study period while the body weight of patients treated with metformin and placebo did not significantly change. Surprisingly, no differences were observed between methazolamide and placebo treated patients who were not using metformin. These newly diagnosed diabetes patients all lost equivalent amounts of weight through dietary modification,

### DETAILED DESCRIPTION

Throughout this specification and the claims which follow, unless the context requires otherwise, the word “comprise” and variations such as “comprises” and “comprising” will be understood to imply the inclusion of a stated integer or step or group of integers but not the exclusion of any other integer or step or group of integers.

The singular forms “a”, “an” and “the” include plural aspects unless the context clearly dictates otherwise.

The term “invention” includes all aspects, embodiments and examples as described herein.

Methazolamide is approved for use in the treatment of ocular conditions where lowering intraocular pressure is likely to be of therapeutic benefit, such as chronic open-angle glaucoma, secondary glaucoma, and preoperatively in acute angle-closure glaucoma where lowering the intraocular pressure is desired before surgery. Although commonly described as a diuretic, it has only a weak and transitory diuretic activity, and product labelling specifically states that it should not be used as a diuretic. Methazolamide exerts its effect on ocular conditions through inhibition of the enzyme carbonic anhydrase; however, this does not appear to be the mechanism responsible for its activity as an insulin sensitizer in diabetes. The therapeutically effective (carbonic anhydrase inhibitory) intraocular pressure-reducing dose of methazolamide is in the range of from 50 mg to 100-150 mg, 2 or 3 times daily, i.e. from 100-450 mg per day. Some metabolic acidosis and electrolyte imbalance may occur with the use of carbonic anhydrase inhibitory effective amounts, but excessive acidosis which, can lead to a symptom complex of malaise, fatigue, weight loss, depression and anorexia, can occur even at dosage amounts at the lower end of the standard dosage range (Epstein and Grant, Arch. Opthamol., 95, 1380, 1977).

In accordance with the disclosure, methazolamide is administered in an amount effective to reduce body weight according to a desired dosing regime. In some embodiments, the amount administered is also sufficient to reduce elevated blood glucose levels or maintain normal or desired blood glucose levels, for example, in a synergistic or additive manner with the anti-diabetic agent. In some embodiments, the body weight reduction effects of methazolamide as disclosed herein can be achieved by dosage amounts such that they avoid or minimise clinically meaningful carbonic anhydrase inhibition, such as required for therapeutic treatment of ocular conditions, and also the dosages used avoid or minimise clinically meaningful acidosis which may be associated with standard carbonic anhydrase inhibitory effective dosage regimes. Thus, in some embodiments, methazolamide is advantageously administered at a dosage rate of less than 100 mg per day. In further embodiments, the methazolamide is administered at a dosage rate of about 90, 80 or 75 mg or less per day, or about 50 mg or less per day. In still further embodiments, the methazolamide is administered at a dosage rate of about 40 mg or less per day. In yet further embodiments the methazolamide is administered at a dosage rate of about 30 mg or less per day. In yet further embodiments the methazolamide is administered at a dosage rate of about 25 mg or less per day. In still further embodiments the methazolamide is administered at a dosage rate of about 20 mg or less per day, such as about 15, 10 or 5 mg per day. Administration of any of these dosage amounts may be once a day, as a single dose, or a divided dose, such as twice or thrice a day or according to any other dosing regime as determined by the attending physician. Suitable unit dosages of methazolamide may contain about 1.0, 2.5, 5.0, 10, 20, 25, 30, 40, 50, 60, 75, 80 or 90 mg of methazolamide.

The patients contemplated herein suffer from a diabetic or pre-diabetic condition, which includes any disease or condition, or symptom or causative factor thereof in which insulin resistance or impaired glucose uptake by a cell or tissue can be attributed, or play a role or is manifested, and for which treatment with an anti-diabetic agent (also referred to herein as an anti-hyperglycemic agent) is prescribed for treatment. Non-limiting examples of diabetic or pre-diabetic conditions, symptoms and causative factors include NIDDM (type 2 diabetes), gestational diabetes, impaired glucose tolerance, impaired fasting glucose, Syndrome X, hyperglycemia, atheriosclerosis, hypertriglyceridemia, dyslipidemia, hyperinsulinemia, nephropathy, neuropathy, ischemia, stroke and fatty liver disease. Typically, the disease or condition is NIDDM, gestational diabetes, impaired glucose tolerance, impaired fasting glucose, Syndrome X or hyperglycemia.

Typically, although not always, patients suffering from or susceptible to a diabetic or pre-diabetic condition have an increased body mass index (BMI). A BMI of 25-29.9 is categorised as “overweight”, or “pre-obese”. A BMI of 30 or greater is categorised as “obese”. Further subcategories define further levels of obesity (obese classes I, II and III) In some embodiments, patients may have a BMI of 25 or greater, e.g. in the range of 25-27, or 27-29.9, or 30-33 or 33-34.9 or greater than 35 or 40. The treatments according to the disclosure may be particularly effective for patients with an increased BMI of 25 or greater or 30 or greater. Increased waist measurement is another risk indicator for type 2 diabetes, the greater the waist measurement the greater the increased risk, and a substantial number of diabetic or pre-diabetic patients may have increased waist measurements (which may be but are not necessarily associated with a BMI of greater than 25). Thus, in some embodiments, patients contemplated herein have a waist measurement of more than 94 or 102 cm (adult men) or more than 80 or 88 cm (adult women).

Patients contemplated by the disclosure have been diagnosed as suffering from or susceptible to conditions as contemplated above and are established on a treatment regime with an anti-diabetic agent (e.g metformin or pharmaceutically acceptable salt thereof-). In some embodiments, said patient has commenced treatment with an anti-diabetic agent at least 1 or 2 weeks prior to commencement of methazolamide treatment. In further embodiments the patient has commenced treatment with an anti-diabetic agent at least 4 weeks (or 1 month) prior to commencement of methazolamide treatment. In still further embodiments the patient has commenced treatment with an anti-diabetic agent at least 6, 8, 10 or 12 weeks (for example at least about 2 or about 3 months) prior to commencement of methazolamide treatment. In some embodiments it is advantageous for the patient to have been stabilised on the anti-diabetic agent prior to commencement of methazolamide treatment, that is to say, a dosing regime has been determined and commenced such that a stable or controlled desired blood glucose level, as determined by the attending physician has been achieved. By way of example, commencing dosages of metformin (as the hydrochloride salt) may be determined by the attending physician and are individualised on the basis of effectiveness and tolerance, generally commencing with once or twice daily doses of 500 or 850 mg per day and adjusted as necessary to achieve stable or controlled blood glucose levels. Once a dosage is established, this may be about 1000-1500 mg per day, up to a maximum dosage of about 2500 mg per day for adult patients. Blood glucose levels can be measured by any suitable means typically used in the art, e.g. fasting blood glucose, HbA_{1c} levels etc. Exemplary stabilised levels include HbA_{1c} levels of less than 6.5% or fasting state blood glucose levels less than about 6.1 mmol/L (110 mg/dL)

Agents for the treatment of associated conditions, such as cardiovascular disease (e.g. antihypertensive agents), may also be administered in conjunction (simultaneously or separately) with the anti-diabetic agent and methazolamide. Any such associated symptoms or conditions may be treated with an appropriate agent, e.g. anti-hypertensives such as diuretics, ACE inhibitors or β-blockers as determined by the attending physician. In some embodiments, the weight loss achieved by the disclosure herein may advantageously obviate the need for or reduce the dosage amount of such agents. It will be understood therefore that a patient may not necessarily suffer from or develop all symptoms or conditions associated with a diabetic or pre-diabetic disease or condition or, the condition may not be severe enough to warrant additional therapeutic treatment particularly if the disease or condition is detected and treated at an early stage.

The methazolamide may be co-administered simultaneously with, or sequentially to (before or after), the anti-diabetic therapeutic agent, and in the case of simultaneous administration, each agent may be formulated separately, or alternatively, both are formulated together into an intimate composition. Suitable anti-diabetic agents may include insulin sensitisers, insulin secretagogues glucose resorption/uptake inhibitors and the classes and compounds identified in US2005/0037981, particularly Table 2, the contents of which are incorporated herein in their entirety. Some examples of agents for use include biguanides, sulfonylureas, meglitinides, insulin and insulin analogues, and thiazolidinediones. Further non-limiting examples include thiazolidinediones (including rosiglitazone and pioglitazone), metformin, insulin, sulphonylureas (including glimepiride, glyburide, glipizide, chlorpropamide, tolazamide and tolbutamide), meglitimides (including repaglinide and nateglinide), a-glucosidase inhibitors (including a carbose and miglitol), GLP analogues such as exenatide and DPPIV inhibitors such as sitagliptin.

In some embodiments, the anti-diabetic agent is an insulin sensitiser. An example thereof is metformin.

In some embodiments, by co-administering methazolamide once the patient is established on a treatment with an anti-diabetic agent, such as metformin, it may be possible to subsequently reduce the dosage of the anti-diabetic agent compared to the initial monotherapy. This may advantageously avoid, ameliorate, or otherwise reduce the severity, risk or occurrence of undesirable side effects and disadvantages associated with dosage amounts and regimes employed for the monotherapy. Thus, in some embodiments, the dosage regime of the anti-diabetic agent commenced prior to methzolamide treatment may be adjusted once methazolamide treatment is commenced or has been undertaken for a period of time.

As used herein, the terms “regulate” or “modulate” and variations such as regulating/modulating and regulation/modulation, when used in reference to glucose homeostasis, refer to the adjustment or control of said glucose levels, in particular embodiments, the adjustment to or maintenance of normal blood glucose levels. Thus, “regulating/modulating glucose homeostasis” includes the adjustment or control of blood glucose levels to lower hyperglycaemic, or advantageously achieve or maintain normal fasting state, blood glucose levels. Normal fasting state blood glucose levels are typically less than 6.1 mmol/L (110 mgd/L). Hyperglycemic levels (also referred to herein as elevated blood glucose levels) refer to fasting blood glucose levels greater than or equal to 6.1 mmol/L (110 mgd/L).

Impaired fasting glycemia (IFG) is characterised by a fasting plasma glucose concentration greater than or equal to 6.1 mmol/L (110 mgd/L) but less than 7.0 (126 mgd/L) and a 2-h plasma glucose concentration during the oral glucose tolerance test (OGTT) (if measured) less than 7.8 mmol/L (140 mgd/L). Impaired glucose tolerance (IGT) is characterised by a fasting plasma glucose concentration of less than 7.0 mmol/L (126 mgd/L) and a 2-h plasma glucose concentration during the OGTT of greater than or equal to 7.8 mmol/L (140 mgd/L) but less than 11.1 mmol/L (200 mgd/L). Diabetes is characterised by a fasting plasma glucose concentration of greater than or equal to 7.0 mmol/L (126 mgd/L) or a 2-h plasma glucose concentration during the OGTT of greater than 11.1 mmol/L (200 mgd/L).

Patients contemplated herein include mammalian subjects: humans, primates, livestock animals (including cows, horses, sheep, pigs and goats), companion animals (including dogs, cats, rabbits, guinea pigs), and captive wild animals. Laboratory animals such as rabbits, mice, rats, guinea pigs and hamsters are also contemplated as they may provide a convenient test system. Human patients are particularly contemplated.

As described above, combinations according to the invention using metformin, or a pharmaceutically acceptable salt thereof, may advantageously allow for reduced dosage amounts of metformin (or pharmaceutically acceptable salt) compared to known metformin therapies, particularly metformin monotherapy. In some embodiments, the dosage amounts of the combinations are such that they may provide an additive or synergistic effect. Suitable dosage amounts and dosing regimens can be determined by the attending physician and may depend on the particular condition being treated, the severity of the condition as well as the general age, health and weight of the subject.

Once methazolamide treatment is commenced, the commencing or established treatment with the anti-diabetic agent may be maintained or further adjusted as necessary. In some embodiments of the invention, the daily dosage amount of the anti-diabetic agent, such as metformin (or pharmaceutically acceptable salt, such as the hydrochloride), administered is decreased. In some embodiments the dosage is adjusted to be equal to or less than about 90% of that is required for the initial or stabilised monotherapy. In further embodiments, the dosage is equal to or less than about 80%, 70%, 60% or 50% of that which would be required for metformin monotherapy. Exemplary daily dosage amounts of metformin for an adult may be in the range of from about 100 mg to about 1500 or 2000 mg of active per day, such as about 250 mg, 500 mg, 750 mg, 850 mg, 1000 mg, 1100 or 1250 mg. Exemplary daily dosage amounts for paediatric patients (10-16 years) may be in the range from about 50, to about 1000 mg or 1500 mg per day, such as about 100 mg, 250 mg, 500 mg, 750 mg, 850 mg, 1100 mg or 1250 mg per day. The anti-diabetic agent may be administered in a single dose or a series of doses. Suitable dosages forms may contain about 50, 75, 100, 150, 200, 250, 500 750, 850 or 1000 mg of metformin.

While methazolamide and the anti-diabetic agent may be administered in the absence of any other agents or additives, it is preferable to present each as a composition with one or more pharmaceutically acceptable additives or together as an intimate composition with one or more pharmaceutically acceptable additives.

The formulation of such compositions is well known to those skilled in the art, see for example, Remington's Pharmaceutical Sciences, 21^{st} Edition. The composition may contain any suitable additives such as carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically acceptable in the sense of being compatible with the other ingredients of the composition and not injurious to the subject. Compositions include those suitable for oral, rectal, inhalable, nasal, topical (including dermal, buccal and sublingual), vaginal or parental (including subcutaneous, intramuscular, intravenous and intradermal) administration. The compositions may conveniently be presented in unit dosage form and may be prepared by any methods well known in the art of pharmacy.

Compositions of the present disclosure suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent), preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, appropriate coatings, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the active ingredients particularly mentioned above, the compositions of this disclosure may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Compounds for administration in accordance with the disclosure may optionally be presented as a pharmaceutically acceptable salt or prodrug as appropriate.

The term “prodrug” is used in its broadest sense and encompasses those derivatives that are converted in vivo, either enzymatically or hydrolytically, to the compounds of the invention. Such derivatives would readily occur to those skilled in the art, and include, for example, compounds where a free thiol or hydroxy group is converted into an ester, such as an acetate, or thioester or where a free amino group is converted into an amide. Procedures for acylating the compounds of the invention, for example to prepare ester and amide prodrugs, are well known in the art and may include treatment of the compound with an appropriate carboxylic acid, anhydride or chloride in the presence of a suitable catalyst or base. Esters of carboxylic acid (carboxy) groups are also contemplated. Suitable esters include C₁₋₆alkyl esters; C₁₋₆alkoxymethyl esters, for example methoxymethyl or ethoxymethyl; C₁₋₆alkanoyloxymethyl esters, for example, pivaloyloxymethyl; phthalidyl esters; C₃₋₈cycloalkoxycarbonylC₁₋₆alkyl esters, for example, 1-cyclohexylcarbonyloxyethyl; 1,3-dioxolen-2-onylmethyl esters, for example, 5-methyl-1,3-dioxolen-2-onylmethyl; and C₁₋₆alkoxycarbonyloxyethyl esters, for example, 1-methoxycarbonyloxyethyl. Prodrugs of amino functional groups include amides (see, for example, Adv. BioSci., 1979, 20, 369, Kyncl, J. et al), enamines (see, for example, J. Pharm. Sci., 1971, 60, 1810, Caldwell, H. et al), Schiff bases (see, for example, U.S. Pat. No. 2,923,661 and Antimicrob. Agents Chemother., 1981, 19, 1004, Smyth, R. et at), oxazolidines (see, for example, J. Pharm. Sci, 1983, 72, 1294, Johansen, M. et al), Mannich bases (see, for example, J. Pharm. Sci. 1980, 69, 44, Bundgaard, H. et al and J. Am. Chem. Soc., 1959, 81, 1198, Gottstein, W. et al), hydroxymethyl derivatives (see, for example, J. Pharm. Sci, 1981, 70, 855, Bansal, P. et al) and N-(acyloxy)alkyl derivatives and carbamates (see, for example, J. Med. Chem., 1980, 23, 469, Bodor, N. et al, J. Med. Chem., 1984, 27, 1037, Firestone, R. et al, J. Med Chem., 1967, 10, 960, Kreiger, M. et al, U.S. Pat. No. 5,684,018 and J. Med. Chem., 1988, 31, 318-322, Alexander, J. et al). Other conventional procedures for the selection and preparation of suitable prodrugs are known in the art and are described, for example, in WO 00/23419; Design of Prodrugs, H. Bundgaard, Ed., Elsevier Science Publishers, 1985; Methods in Enzymology, 42: 309-396, K. Widder, Ed, Academic Press, 1985; A Textbook of Drug Design and Development, Krogsgaard-Larsen and H. Bundgaard, Eds, Chapter 5, p 113-191 (1991); Advanced Drug Delivery Reviews, 8; 1-38 (1992); Journal of Pharmaceutical Sciences, 77; 285 (1988), H. Bundgaard, et al; Chem Pharm Bull, 32692 (1984), N. Kakeya et al and The Organic Chemistry of Drug Desig and Drug Action, Chapter 8, pp 352-401, Academic press, Inc., 1992.

Suitable pharmaceutically acceptable salts include, but are not limited to salts of pharmaceutically acceptable inorganic acids such as hydrochloric, sulphuric, phosphoric nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic, fendizoic, 4-4′-methylenebis-3-hydroxy-2-naphthoic acid, 0-(p-hydroxybenzoyl)benzoic, 4′-4″-dihydroxytriphenylmethane-2-carboxylic acid and valeric acids. Base salts include, but are not limited to, those formed with pharmaceutically acceptable cations, such as sodium, potassium, lithium, calcium, magnesium, ammonium and alkylammonium. Basic nitrogen-containing groups may be quaternised with such agents as lower alkyl halide, such as methyl, ethyl, propyl, and butyl chlorides, bromides and iodides; dialkyl sulfates like dimethyl and diethyl sulfate; and others.

The compounds of the invention may also be presented for use in veterinary compositions. These may be prepared by any suitable means known in the art. Examples of such compositions include those adapted for:
(a) oral administration, e.g. tablets, boluses, powders, granules, pellets for admixture with feedstuffs, pastes for application to the tongue, drenches including aqueous and non-aqueous solutions or suspensions;
(b) parenteral administration, e.g. subcutaneous, intramuscular or intravenous injection as a sterile solution or suspension.

The invention will now be described with reference to the following example which is provided for the purpose of illustrating an embodiment of the invention and is not to be construed as limiting the generality hereinbefore described.

### EXAMPLE

The safety and efficacy of methazolamide (40 mg administered twice daily) as a potential treatment for type 2 diabetes were evaluated in a 24 week, randomised, placebo-controlled double-blind clinical trial. The primary efficacy endpoint for the clinical trial was a reduction in HbA_{1c} (ΔHbA_{1c}) from baseline with methazolamide, relative to placebo, after 24 weeks of treatment. Secondary efficacy endpoints included reduction in body weight and improvements in cardiovascular measures such as blood pressure. The primary safety measurement was the effect of methazolamide, compared to placebo, on venous blood gas parameters; a measure of acidosis.

The clinical trial initially enrolled type 2 diabetes patients who were not treated with any anti-diabetic agent prior to entry into the trial (NAIVE) The trial was expanded to include participants who had been treated with metformin for at least 3 months and were on a stable metformin dose for at least 8 weeks prior to entering the trial (MET). The metformin dose was not altered throughout the trial. Participant baseline demographic data are provided in Table 1,

Participants randomized into the clinical trial were administered either daily doses of methazolamide (40 mg b.i.d.) or placebo for 24 weeks. Methazolamide was taken as 1×30 mg capsule and 1×10 mg capsule per dose at breakfast and dinner. Placebo (microcrystalline cellulose) was administered in identical presentation. After an initial randomization visit to the clinic (Day 0), participants returned to the clinic at weeks 1, 2, 4, 8, 12, 18 and 24 for physical examinations, laboratory analyses, body composition measures, evaluation of glycemic parameters (fasting blood glucose, fasting insulin, HbA_{1c}) and measurement of venous blood gas analysis.

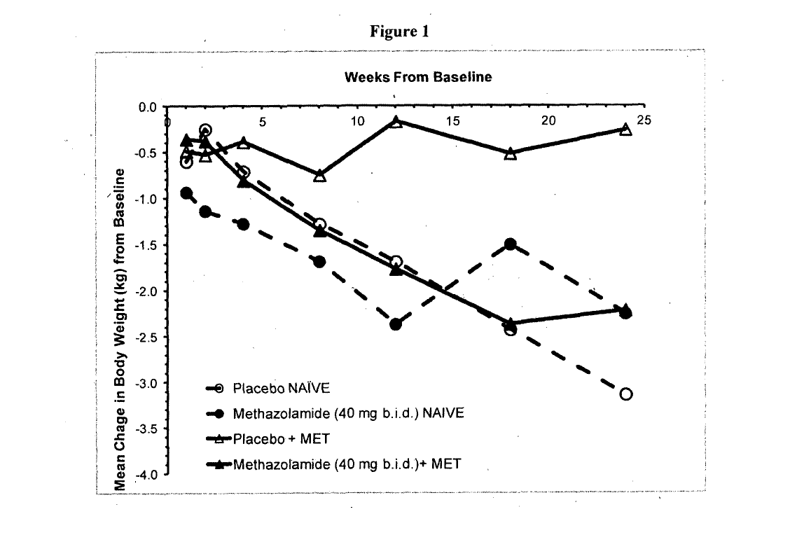

The effects of methazolamide and placebo on HbA_{1c}, body weight, and BMI are presented in Table 2. Mean changes in body weight over time are depicted in FIG. 1.

Metformin-treated patients who received placebo maintained a largely stable body weight throughout the study. Surprisingly, metformin-treated patients further treated with methazolamide lost an average of 2.2 kg over the 24 week period, which is 2% of their starting body weight. No such additional effect of methazolamide was observed in Naïve patients, where both methazolamide and placebo groups lost weight due to changes in diet typical of newly diagnosed diabetes patients. Thus an unexpected and selective effect of methazolamide on the metformin-treated patients was observed.

TABLE 1
Baseline (day 0) demographic data for methazolamide (MTZ) clinical; trial
participants

| Parameter | Placebo Alone | MTZ Alone | Placebo + Met | MTZ + Met |
|---|---|---|---|---|
| No. | 20 | 15 | 19 | 22 |
| Male (female) | 9 (11) | 10 (5) | 13 (6) | 18 (4) |
| Age (yr) | | | | |
| Mean ± SD | 64 ± 8 | 63 ± 10 | 61 ± 10 | 63 ± 9 |
| Median (range) | 65 (51-76) | 65 (32-75) | 62 (35-76) | 64 (45-76) |
| Body weight (kg) | | | | |
| Mean ± SD | 90.2 ± 17.6 | 93.0 ± 13.7 | 90.5 ± 14.9 | 92.3 ± 15.1 |
| Median (range) | 95.1 (57.2-123.0) | 95.3 (65.6-107.4) | 89.9 (69.0-130.0) | 89.6 (67.4-124.0) |
| Height (cm) | | | | |
| Mean ± SD | 168 ± 10 | 170 ± 8 | 174 ± 9 | 172 ± 7 |
| Median (Range) | 169 (145-185) | 170 (158-182) | 173 (158-195) | 171 (155-184) |
| BMI (kg/m²) | | | | |
| Mean ± SD | 31.8 ± 3.9 | 32.3 ± 4.9 | 30.0 ± 4.4 | 31.4 ± 5.1 |
| Median (range) | 32.0 (23.8-38.1) | 32.3 (25.3-39.5) | 29.4 (23.3-38.4) | 29.9 (24.1-39.7) |
| Waist (cm) | | | | |
| Mean ± SD | 105 ± 13 | 107 ± 11 | 102 ± 10 | 107 ± 12 |
| Median (range) | 105 (79-125) | 109 (90-127) | 101 (81-122) | 102 (90-131) |
| Metformin (mg/day) | | | | |
| Mean ± SD | — | — | 1387 ± 642 | 1545 ± 999 |
| Median (range) | — | — | 1000 (500-3000) | 1250 (500-4500) |
| HbA_{1c} (%) | | | | |
| Mean ± SD | 7.2 ± 0.6 | 7.1 ± 1.0 | 7.6 ± 0.5^{b} | 7.2 ± 0.4 |
| Median (range) | 7.15 (6.4^{c}-8.3) | 6.7 (6.2^{c}-10.1^{d}) | 7.7 (6.7-8.4) | 7.1 (6.6-8.0) |

^{a}n = 38.
^{b}n = 18.
^{c}HbA_{1c} = 6.5% at screening visit prior to randomization.
^{d}HbA_{1c} 8.4% at screening visit prior to randomization.
MTZ = methazolamide;
Met = metformin

TABLE 2
HbA_{1c}, body weight (BW) and body mass index (BMI) and changes in these
parameters from baseline (Day 0) to Week 24 (ΔHbA_{1c}, ΔBW, ΔBMI)

| Parameter | Placebo Alone | MTZ Alone | Placebo + Met | MTZ + Met |
|---|---|---|---|---|
| HbA_{1c} Day 0 (%) | | | | |
| n | 20 | 15 | 18 | 22 |
| Mean ± SD | 7.16 ± 0.56 | 7.09 ± 0.99 | 7.58 ± 0.53 | 7.16 ± 0.38 |
| Median (range) | 7.15 (6.4, 8.3) | 6.7 (6.2, 10.1) | 7.70 (6.7, 8.4) | 7.1 (6.6, 8.0) |
| HbA_{1c} Week 24 | | | | |
| n | 20 | 13 | 16 | 20 |
| Mean ± SD | 7.31 ± 1.61 | 6.88 ± 0.63 | 7.50 ± 0.84 | 6.91 ± 0.61 |
| Median (range) | 7.0 (6.2, 13.5) | 7.0 (6.1, 8.0) | 7.3 (6.6, 9.8) | 7.0 (5.6, 8.2) |
| ΔHbA_{1c} Week 24 | | | | |
| n | 20 | 13 | 16 | 20 |
| Mean ± SD | +0.16 ± 1.31 | −0.26 ± 0.72 | −0.04 ± 0.71 | −0.25 ± 0.67 |
| Median (range) | −0.15 (−1.0, +5.2) | −0.3 (−2.1, +0.8) | −0.2 (−0.9, +1.4) | −0.15 (−1.6, +0.8) |
| MTZ-Placebo | | −0.1 | | −0.21 |
| BW Baseline (kg) | | | | |
| n | 20 | 15 | 19 | 22 |
| Mean ± SD | 90.2 ± 17.6 | 93.0 ± 13.7 | 90.5 ± 14.9 | 92.3 ± 15.1 |
| Median (range) | 95.1 (57.2, 123.0) | 95.3 (65.6, 107.4) | 89.9 (69.0, 130.0) | 89.6 (67.4, 124.0) |
| BW Week 24 | | | | |
| N | 20 | 15 | 18 | 21 |
| Mean ± SD | 87.1 ± 16.7 | 90.7 ± 14.0 | 90.3 ± 15.9 | 89.0 ± 14.3 |
| Median (range) | 88.7 (57.2, 122.1) | 95.3 (63.3, 106.0) | 87.6 (68.5, 133.6) | 87.2 (64.8, 127.0) |
| ΔBW Week 24 | | | | |
| N | 20 | 15 | 18 | 21 |
| Mean ± SD | −3.1 ± 3.1 | −2.3 ± 2.4 | −0.3 ± 1.7 | −2.2 ± 3.6* |
| Median (range) | −2.65 (−10.3, +0.4) | −1.8 (−9.8, +1.3) | −0.5 (−2.8, +3.9) | −2.0 (−15.3, +3.0) |
| MTZ-Placebo | | +0.8 | | −1.9 |
| BMI Day 0 (kg/m²) | | | | |
| n | 20 | 15 | 19 | 22 |
| Mean ± SD | 31.8 ± 3.9 | 32.3 ± 4.9 | 30.0 ± 4.4 | 31.4 ± 5.1 |
| Median (range) | 32.0 (23.8-38.1) | 32.3 (25.3-39.5) | 29.4 (23.3-38.4) | 29.9 (24.1-39.7) |
| BMI Week 24 | | | | |
| n | 20 | 15 | 18 | 21 |
| Mean ± SD | 30.6 ± 3.6 | 31.5 ± 5.1 | 29.7 ± 4.7 | 30.3 ± 4.8 |
| Median (range) | 30.85 (23.8-35.7) | 32.0 (24.7-39.0) | 29.1 (23.6-39.5) | 29.8 (23.0-39.7) |
| ΔBMI Week 24 | | | | |
| n | 20 | 15 | 18 | 21 |
| Mean ± SD | −1.17 ± 1.12 | −0.77 ± 0.79 | −0.06 ± 0.58 | −0.78 ± 1.21* |
| Median (range) | −1.00 (−3.50, 0.00) | −0.6 (−3.1, +0.5) | −0.2 (−0.8, +1.5) | −0.6 (−5.1, −0.9) |
| MTZ-Placebo | | +0.4 | | −0.72 |

*p < 0.05 vs. Placebo + MET (unpaired 2-sided t-test).
MTZ = methazolamide;
Met = metformin

1-12. (canceled)

13. A method of effecting weight loss in a patient comprising:
commencing treatment with metformin;
continuing treatment with metformin for at least 3 months;
subsequently commencing additional treatment with methazolamide in an amount of 80 mg/day; and
continuing treatment with methazolamide for at least 24 weeks,
wherein the patient has type 2 diabetes and a body mass index of at least 23 kg/m² and experiences a greater weight loss than patients undergoing the same method of treatment except for receiving placebo instead of methazolamide.

14. The method of claim 13, wherein the continuing treatment with metformin comprises treatment with a stable dose of metformin for at least 8 weeks.

15. The method of claim 14, wherein the dose of metformin is 500-4500 mg/day.

16. The method of claim 14, wherein the patient's blood glucose levels are stabilized by metformin.

17. The method of claim 13, wherein the methazolamide and metformin are administered orally, either simultaneously or separately.

18. The method of claim 13, wherein the methazolamide is divided into two doses.

19. The method of claim 13, wherein the patient has a body mass index of at least 30 kg/m².

20. The method of claim 13, wherein the patient has a waist measurement of greater than 94 cm (adult men) or greater than 80 cm (adult women).

21. The method of claim 13, wherein the HbA_{1c} level of the patient decreases compared to patients undergoing the same method of treatment except for receiving placebo instead of methazolamide.

22. The method of claim 13, wherein the body mass index of the patient decreases compared to patients undergoing the same method of treatment except for receiving placebo instead of methazolamide.
